(19) United States
(12) Patent Application Publication
(10) Pub. No.: US 2005/0232994 A1
(43) Pub. Date: Oct. 20, 2005

(54) Dual-spike release formulation for oral drug delivery

(76) Inventor: Bernard Charles Sherman, Toronto (CA)

Correspondence Address: IVOR M. HUGHES, BARRISTER & SOLICITOR,;PATENT & TRADEMARK AGENTS, 175 COMMERCE VALLEY DRIVE WEST, SUITE 200, THORNHILL, ON, L3T 7P6, CA

(21) Appl. No.: 10/523,761

(22) PCT Filed: Jun. 8, 2003
(86) PCT No.: PCT/CA03/01175

§ 371 Date: Feb. 7, 2005

(30) Foreign Application Priority Data
Aug. 13, 2002 (CA) 2,395,819

(51) Int. Cl. A61K 31/445; A61K 9/24

(52) U.S. Cl. 424/471; 514/317

(57) ABSTRACT

A composition which achieves release of a drug in two spikes, and which comprises particles, wherein the particles consist of a homogenous mixture which comprises the drug and an enteric polymer.

### BACKGROUND OF THE INVENTION

Methylphenidate is a compound used to treat hyperactivity and attention deficit disorder (ADD) in children.

Methylphenidate is sold in the United States and elsewhere under the trade name Ritalin™. More particularly, it is sold as Ritalin™ immediate-release tablets for oral administration containing methylphenidate as the hydrochloride salt in strengths of 5, 10 and 20 mg. The drug is also available in Ritalin SR™ tablets which contain methylphenidate as the hydrochloride salt in strength of 20 mg in a slow-release formulation.

Ritalin SR™ is not suitable for optimal treatment of ADD because it produces a continuous slow release, whereas methylphenidate requires a spike or “sawtooth” (i.e. multi-spike) profile to have maximum efficacy in the treatment of ADD. For this reason, ADD has usually been treated with prompt release tablets given two or three times daily.

It would thus be desirable to have a dosage form that could be administered as a single dose in the morning and would release the drug in two spikes at least one hour apart.

Such a product is now being introduced into the United States market under the tradename Ritalin LA™ capsules in strengths of 20, 30 and 40 mg. These capsules are made in accordance with the teachings of U.S. patent application number U.S. 2001/0046472. Each capsule contains two different types of beads. Half of the dose is in immediate-release beads, which release their drug content in the stomach. The other half of the dose is in delayed-release beads. These are beads which are enteric coated; that is to say they are coated with a polymer that is insoluble at acidic gastric pH, but soluble at intestinal pH. Hence, the delayed-release beads release these drug contents only after the pellets reach the small intestine and the enteric coating dissolves.

While the formulation of Ritalin LA capsules achieves the desired result of two spikes, the formulation is complex and expensive to manufacture. It requires production of two different types of beads, and also requires capsule-filling equipment that is capable of filling two different types of beads into a single capsule.

In view of this prior art, the objective of the present invention is to enable a formulation that will release the drug content in two peaks from a single type of bead, pellet or granule.

### DESCRIPTION OF THE INVENTION

It has been found that particles in the form of beads, pellets or granules can be made of a single type that release a drug in two spikes, the first spike occurring promptly upon the particles reaching the stomach, and the second peak occurring after the particles have traveled into the small intestine.

The present invention is a pharmaceutical composition comprising such particles.

The particles consist of an essentially homogenous mixture, which comprises both a water-soluble drug and an enteric polymer. It has been found that if the particles are very small and the ratio of enteric polymer to the drug is low, then all or essentially all of the drug will be promptly released in the stomach. However, as the size of the particles is increased and the ratio of enteric polymer to drug is increased, it is found that only the drug content that is closest to the surface of the particles leaches out of the mixture and dissolves in the acidic gastric fluid; dissolution then ceases, as the portion of the drug that is further into the particles is protected against dissolution by the remaining enteric polymer in the outer portion of the particle from which the drug has leached. Dissolution then essentially ceases until the particles reach the small intestine and the pH is high enough so that the enteric polymer dissolves to release the balance of the drug.

By trial and error, a ratio of the enteric polymer to drug and a particle size range may be found such that approximately half of the drug will be promptly released in the stomach and the other half will be released in the small intestine.

The ratio of enteric polymer to drug by weight will preferably be at least 2 and less than 100, will more preferably be from about 4 to about 50 and will most preferably be from about 10 to about 20.

The drug may be methylphenidate or a salt thereof, preferably methylphenidate hydrochloride.

The enteric polymer may be any enteric polymer acceptable for use in pharmaceuticals such as, for example, polyvinyl acetate phthalate, hydroxypropyl methylcellulose phthalate, cellulose acetate phthalate, cellulose acetate trimethitate, hydroxypropyl methylcellulose acetate succinate, and methyacrylic acid copolymer type C. Most preferred is polyvinyl acetate phthalate.

The invention will be better understood from the following example, which is intended to be illustrative and not intended to limit the scope of the invention.

Drug and enteric polymer were mixed in the following proportions.

| | |
|---|---|
| Methylphenidate Hydrochloride | 20 parts |
| Polyvinyl acetate phthalate | 260 parts |
| | 280 parts |

The mixture was compressed into slugs (large tablets) on a tablet press. The slugs were then ground up through #8 screen (8 wires to the inch). The resulting granules were then shaken on a #16 screen, and the portion that went through the #16 screen was discarded. The granules that remained on the screen were then filled into capsules at a net fill of 280 mg per capsule, so that each capsule contained granules comprising 20 mg of methylphenidate hcl.

Dissolution of the capsules was then tested in United States Pharmacopoeia apparatus #2 at 50 RPM, in both simulated gastric fluid and simulated intestinal fluid of pH6.8. It was found that, in simulated gastric fluid, approximately half of the drug was released promptly (within 30 minutes) and dissolution then ceased.

On the other hand, in simulated intestinal fluid, dissolution was essentially complete within 2 hours. It follows that when these capsules are ingested, approximately half of the drug content will be released promptly in the stomach, and the rest will be released within about 2 hours after the particles reach the more alkaline fluid of the small intestine.

1. A composition for oral administration which achieves drug release in two spikes, said composition comprising particles of a homogenous mixture of methylphenidate or a salt thereof and an enteric polymer, wherein the ratio of enteric polymer to methylphenidate or a salt thereof is greater than 4 and less than 100.

2. The composition of any of claims 1 wherein the enteric polymer is polyvinyl acetate phthalate.

3. The composition of any of claims 1, or 2, wherein the ratio of enteric polymer to drug is greater than 4 to about 50.

4. The composition of any claim 1, or 2 wherein the ratio of enteric polymer to drug is from about 10 to about 20.

5. The composition of any previous claim wherein said particles of the composition further comprise granules sized so as to pass through a #8 mesh screen but not pass through a #16 mesh screen.

6. The composition any previous claim wherein some of the drug is released promptly, after ingestion, when the composition reaches the stomach and release of the balance is delayed until the particles reach the small intestinal.
